(19) United States
(12) Patent Application Publication
(10) Pub. No.: US 2004/0210162 A1
(43) Pub. Date: Oct. 21, 2004

(54) Unitary blood sampling apparatus and method of using same

(76) Inventors: Philip W. Wyatt, La Canada, CA (US); Gary Schaeffer, Arcadia, CA (US)

Correspondence Address: James E. Brunton, Esquire, Suite 860, 700 N. Brand Blvd., Post Office Box 29000, Glendale, CA, 91203, US

(21) Appl. No.: 10/420,396

(22) Filed: Apr. 21, 2003

(51) Int. Cl. A61B 5/00

(52) U.S. Cl. 600/573

(57) ABSTRACT

A blood sampling apparatus that includes a low dead space-sampling device, which is of a unitary construction that can be interposed as a single unit into an infusion line between a source of fluid and the patient. The unitary sampling device includes an elongated body having a fluid passageway there through, the fluid passageway having first and second ends, a stopcock assembly connected to the elongated body intermediate the first and second ends and a sampling port assembly having a body portion that is integrally formed with the elongated body intermediate the stopcock assembly and the second end of the elongated body.

### BACKGROUND OF THE INVENTION

1. Field of the Invention

The present invention relates generally to blood sampling apparatus. More particularly the invention concerns a unitary blood sampling apparatus and the method of using same for obtaining precise volumes of substantially undiluted blood for use in blood gas analysis and related therapeutic techniques.

2. Discussion of the Prior Art

Over the years, the techniques for blood sampling have been modified and improved so that they now provide clinicians with a valuable tool useful for obtaining laboratory specimens of arterial/venous blood from intravenous and/or pressure monitoring lines which are interconnected invasively to the patient. For example, the increased use of indwelling arterial/venous catheters has allowed clinicians to take advantage of the easy access to the intra-arterial/venous lines for blood sampling. Accordingly, present practice is to draw substantially all blood specimens from the intra-arterial/venous lines when they are used, thereby decreasing the number of venipunctures required.

Several products and procedures presently exist for use in drawing fluids from arterial/venous monitoring systems. One current method uses an idle side port of a three-port stopcock, which has been interconnected within the intra-arterial/venous line that has been interconnected invasively to the patient. Other methods involve the use of a variety of types of commercially available “T-connectors” or “heparin lock” injection/aspiration sites which may be attached at the side port of a three port stopcock disposed within the most frequently used types of pressure monitoring systems or inline between the arterial/venous catheter and the pressure monitoring line.

One of the most successful methods and apparatus for arterial and venous blood sampling is disclosed in U.S. Pat. No. 4,981,140 issued to one of the present inventors. However, unlike the apparatus of the present invention the prior art apparatus disclosed in this earlier patent does not include a unitary blood sampling device that can be interposed into the intra-arterial/venous line, but rather is made up of a number of separate components that must be strategically interconnected within the line that is invasively connected to the patient.

The thrust of the present invention is to uniquely overcome the various drawbacks of the foregoing art blood sampling techniques, which drawbacks will be discussed in greater detail in the paragraphs, which follow.

### SUMMARY OF THE INVENTION

An object of the present invention is to provide a method and apparatus that will effectively eliminate the clinical problems now associated with arterial and venous blood sampling methods and apparatus. More particularly it is an object of the invention to eliminate problems such as air entry and line contamination associated with the opening of closed monitoring systems, sample dilution and excessive blood waste, and the inability to effectively purge lines of either air, heparinized solution or residue blood.

Another object of the invention is to provide a novel, low dead space-sampling device, which is of a unitary construction that can be interposed as a single unit into an infusion line between a source of fluid and the patient.

Another object of the invention is to provide a novel, low dead space-sampling device of the aforementioned character that is easy to use and effectively prevents line contamination.

Still another object of the invention is to provide a method and apparatus of the aforementioned character, which is safe, reliable and highly cost effective.

### BRIEF DESCRIPTION OF THE DRAWINGS

FIG. 1 is a schematic representation of a typical prior art pressure monitoring apparatus.

FIG. 2 is a greatly enlarged, side elevational, cross-sectional view of one of the stopcocks, or valves, used in the prior art apparatus of FIG. 1.

FIG. 3 is a side elevational view of one form of a unitary blood-sampling device of the present invention.

FIG. 4 is a cross-sectional view taken along lines 4-4 of FIG. 3.

FIG. 5 is a cross-sectional view of one form of a connector component of the invention shown interconnected with a sampling port of the character shown in FIGS. 3 and 4.

FIG. 6 is a plan view of the stopcock component of the sampling device shown in FIG. 3 illustrating one fluid flow path through the stopcock.

FIG. 7 is a plan view of the stopcock component of the sampling device shown in FIG. 3 illustrating another fluid flow path through the stopcock.

FIG. 8 is a plan view of the stopcock component of the sampling device shown in FIG. 3 illustrating still another fluid flow path through the stopcock.

FIG. 9 is a generally perspective view of one form of the blood sampling apparatus of the invention that includes an alternate form of the unitary blood sampling device and an alternate form of medical connector that can be interconnected with the sampling port of the device.

FIG. 10 is an enlarged, cross-sectional view taken along lines 10-10 of FIG. 9.

FIG. 11 is a top plan view of an alternate form of sampling device of the invention.

FIG. 12 is an enlarged, cross-sectional view taken along lines 11-11 of FIG. 11.

FIG. 13 is a cross-sectional view similar to FIG. 12, but showing an alternate form of sampling port of the apparatus of the invention.

FIG. 14 is a view taken along lines 14-14 of FIG. 13.

FIG. 15 is a top plan view, partly in cross section of yet another form of the sampling device of the present invention.

FIG. 16 is a top plan view of yet another embodiment of the invention.

FIG. 17 is a cross-sectional view taken along lines 17-17 of FIG. 16.

FIG. 18 is a cross-sectional view similar to FIG. 6, but showing an alternate form of stopcock component having a curved flow passageway.

FIG. 19 is a cross-sectional view similar to FIG. 18, but showing the stopcock moved to a second position.

FIG. 20 is a cross-sectional view similar to FIG. 19, but showing the stopcock rotated to a third position.

### DESCRIPTION OF THE INVENTION

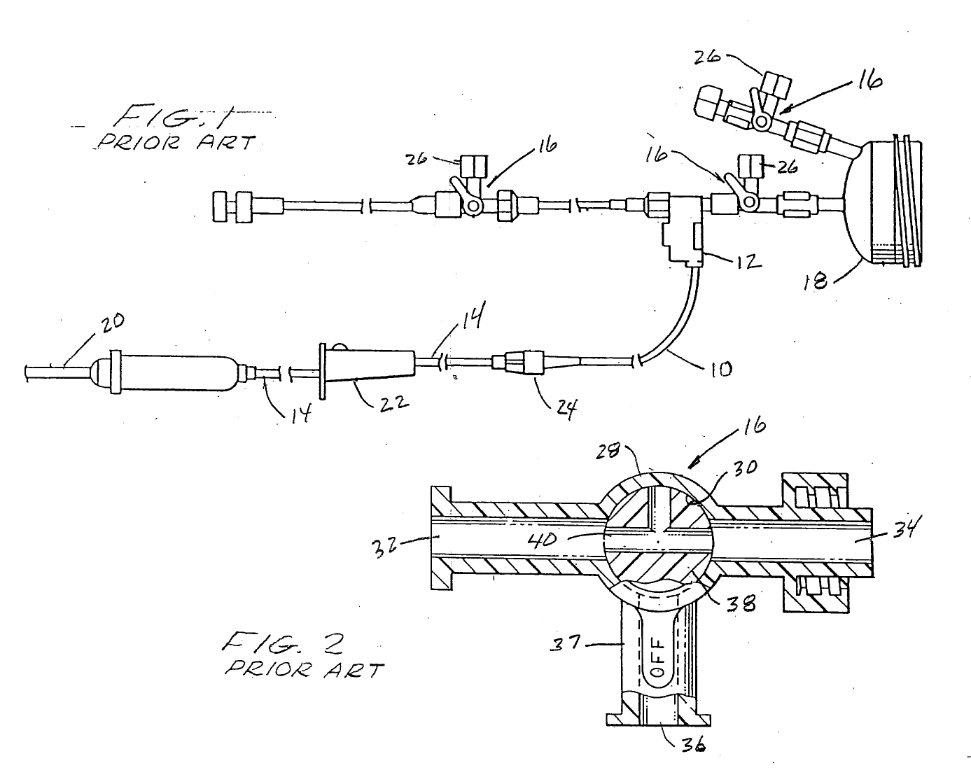

Referring to the drawings and particularly to FIGS. 1 and 2, one form of prior art sampling system is there shown. This system comprises an intravenous (IV) administration set 10, a continuous flush device (CFD) 12, several lengths of IV or pressure tubing 14, a plurality of stopcocks 16, a pressure transducer-dome set 18, and either an arterial or venous catheter (one to five lumens). Generally the entire monitoring system is filled with heparinized solution to maintain patency in the line and to provide the media for transmission of the patient's cardiac pressure to the monitoring equipment.

Typically the IV administration set 10 includes a spike 20, a roller clamp 22 and suitable tube connectors 24. In FIG. 1, the stopcocks 16 are shown in the all closed position and the side ports are shown sealed against atmosphere by port protectors, or dead end caps, 26.

Turning to FIG. 2, the prior art stopcock 16 that is interconnected within the system comprises a body 28 having a central chamber, or core, 30 in communication with inlet, outlet and side port fluid passages 32, 34 and 36 respectively. A flow control member 38 having a “T” shaped fluid passageway 40 is rotatably carried within central chamber 30 and functions to selectively control the flow of fluid through the various fluid passageways of the stopcock. As shown in FIG. 1, lengths of tubing 14 are interconnected with the inlet and outlet fluid passageways 32 and 34 of the stopcock.

When the prior art “stopcock sampling method” is used, the side port 37 of the three-port stopcock provides access to the monitoring line for blood sampling. This “stopcock” technique commonly comprises the following steps:

(1) turning the stopcock control member 38 in a manner to close off side port passageway 36 (FIG. 2);

(2) removing the protector cap 26;

(3) aseptically wiping the side port surfaces with a sterile solution such as alcohol or iodine solution;

(4) substituting a sterile syringe (not shown) for the protector cap;

(5) rotating the stopcock control member, including the handle, to a first position where it blocks CFD flow via passageway 32 while opening passageway 34 to the patient and passageway 36 to the syringe;

(6) aspirating all the heparin or flush solution (plus some amount of blood) from the line leading to the patient and causing the line to fill completely with the patient's blood;

(7) turning the stopcock control member to a second position, midway between passageway 32 and passageway 36, which blocks flow through all passageways;

(8) removing and discarding the syringe with the extracted heparin solution and some waste blood;

(9) attaching a new sterile syringe to the stopcock side port 37 and returning the stopcock control member to its first position;

(10) aspirating the blood specimen into the syringe and then turning the stopcock control member to close fluid flow to the syringe via passageway 36 while reopening the heparin flow to the patient via passageways 32 and 34;

(11) removing the syringe and forwarding the blood sample for laboratory analysis;

(12) activating the CFD to purge the remaining blood in the intra-arterial/venous line back into the patient;

(13) turning the stopcock control member so as to block fluid flow in the patient's direction via passageway 32 while opening the fluid flow from heparin source to the stopcock side port;

(14) again activating the CFD to purge the residue blood from stopcock side port; and

(15) turning the stopcock control member one last time to continue fluid flow communication between heparin source and patient via passageways 32 and 34 while blocking fluid flow to the side port passageway 36 and finally attaching the port protector 26 to the side port.

The “stopcock” method as described in the preceding paragraphs presents several serious problems. For example, the repeated invasion of the normally atmospherically sealed arterial/venous line using stopcocks is a major source for nosocomial infection both in the actual blood sampling steps as well as in the steps involving the handling or replacement of port protectors. Another serious problem inherent in the “stopcock” method involves the unavoidable entry of air into the sample via side port passageway 36 during the sampling steps thereby causing potential error in the sample analysis and in pressure measurements. Additionally, use of the “stopcock” procedure requires that the clinician unavoidably discard a small amount of blood in order to acquire nondiluted blood samples. Where absolute minimal samples must be withdrawn, as is the case with neonates, blood waste is extremely critical and often unacceptable since the waste blood cannot be replaced. Finally error in stopcock manipulation or failure to properly cap open ports can also lead to gross blood loss and catastrophic results.

Because of the serious problem inherent in the “stopcock” sampling method, various modifications of the above procedure have been suggested. For example, two stopcocks inline have been suggested to prevent opening the system to air. However, due to the fact that stopcocks inherently embody large dead space volumes, as for example in one leg of the fluid flow control member, this procedure unavoidably results in unacceptable dilution of the fluid sample and results in serious errors in analysis determination.

The prior art blood sampling techniques which involve the use of heparin locks and “T” connectors are discussed in the previously identified in U.S. Pat. No. 5,603,706.

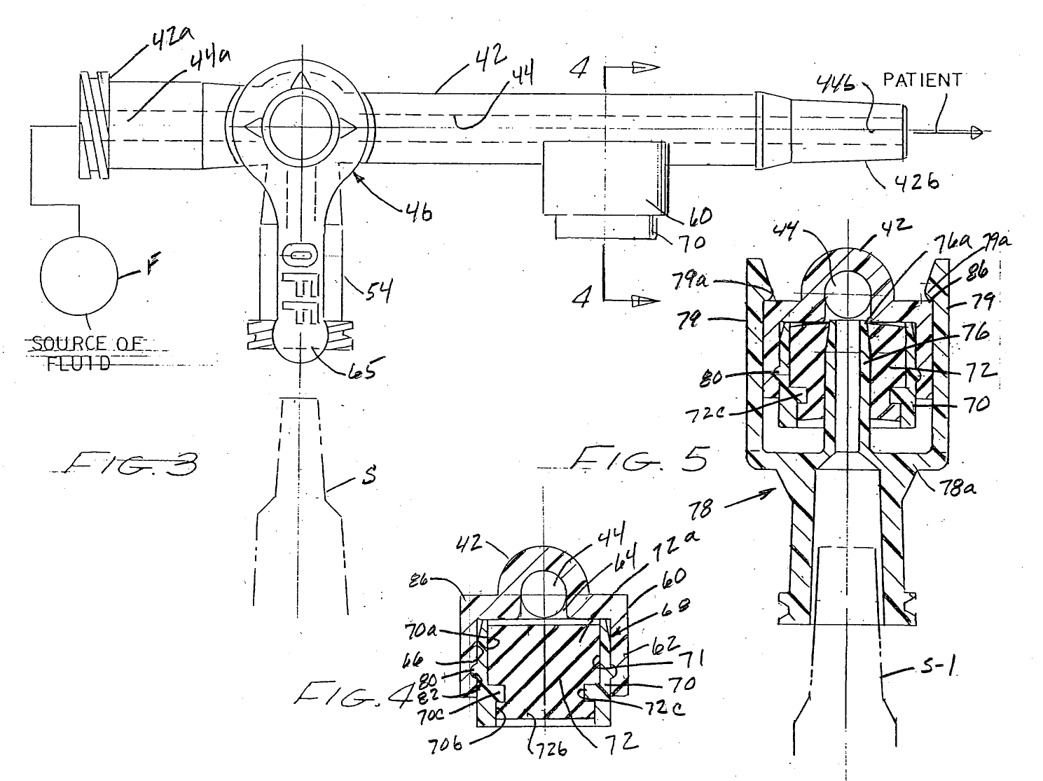

Referring now to FIGS. 3 and 4 of the drawings, one form of the unitary blood sampling device of the invention is there shown and can be seen to comprise an elongated, unitary plastic body 42 having a fluid passageway 44 there t-rough. Plastic body 42, which is fabricated for use as a single piece, includes, proximate either end, connector portions 42a and 42b. Integrally formed with the elongated body 42 intermediate the first and second ends 44a and 44b of fluid passageway 44 is a novel stopcock assembly 46. As best seen in FIG. 6, stopcock assembly 46 includes a body portion 48 having a central chamber 48a that is in communication with fluid passageway 44 of elongated body 44. Body portion 48 of the stopcock assembly further includes an inlet 50, an outlet 52 and a syringe accessible side port 54, each of which is in communication with central chamber 48a. As indicated in FIGS. 3 and 6, syringe accessible side port 54 includes a tapered bore 54a that sealably receives the end portion of a conventional syringe “S” (such as is shown in FIG. 3) so that fluid contained within passageway 44 can be controllably withdrawn into the syringe in a manner well understood by those skilled in the art.

Rotatably mounted within central chamber 48a of the body portion 48 is a flow control member 56 that is provided with a generally “T” shaped fluid passageway 56a for selectively controlling the flow of fluid through the device upon rotation of the flow control member 56 within central chamber 48a. Rotation of member 56 is accomplished by rotating the stopcock handle 65 in the manner illustrated in FIGS. 6 through 8. It is to be noted that the side and bottom walls of central chamber 48 smoothly mate to the inside diameter of passageway 44 to provide nonturbulent, substantially laminar fluid flow and to substantially, if not completely, eliminate space volume.

Also forming an important part of the integral blood sampling device of the present invention is a low dead space sampling port assembly 60 that is integrally formed with elongated body 42 intermediate stopcock assembly 46 and said second end 44b of fluid passageway 44. As best seen in FIG. 4, this novel sampling port assembly 60 comprises a sampling port body 62 that it is integrally formed with elongated body 42. In the present form of the invention sampling port body 62 includes the first and second chambers 64 and 66 respectively. As illustrated in FIG. 4, the first or upper chamber 64 is provided with smoothly curved bottom and sidewalls and is in direct communication with fluid passageway 44 of elongated body 42. The unitary blood-sampling device of the invention is preferably constricted from a rigid, plastic polycarbonate material, although other plastic materials can be used in its fabrication. It is important to note that the entry and exit paths into fluid chamber 48a of the stopcock assembly and into chamber 64 of sampling port body 60 smoothly mate tangently to the inside diameter of passageway 44 to provide nonturbulent fluid flow and to significantly minimize dead space volume.

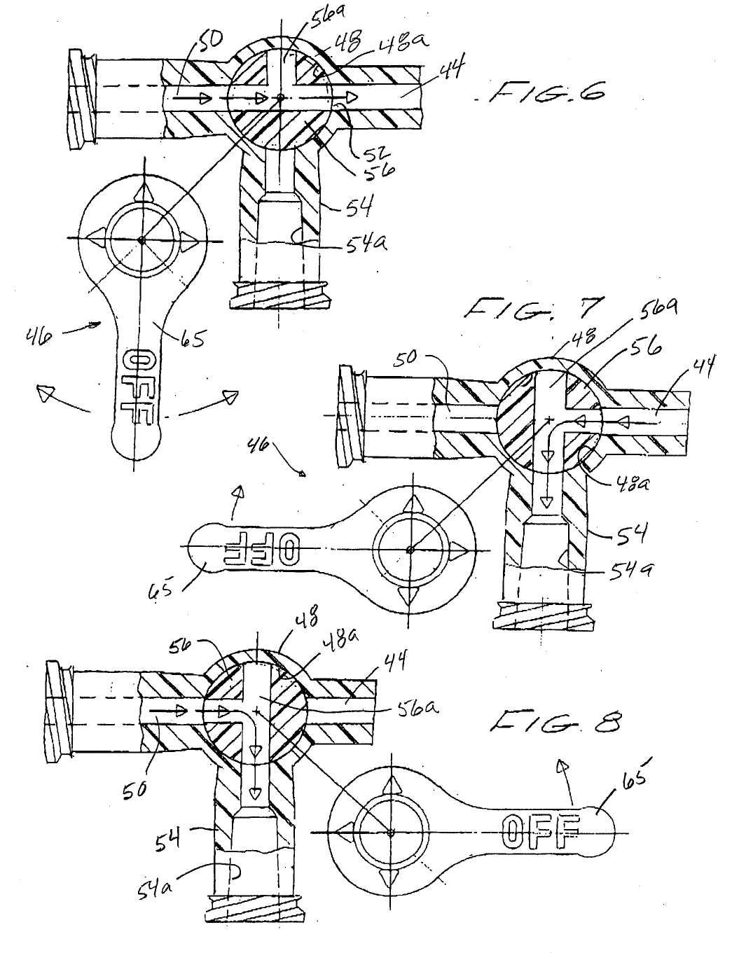

Also forming a part of the sampling port assembly 60 is closure means for securely sealing upper chamber 64 relative to atmosphere. This important closure means is shown here as a closure assembly 68 that comprises a septum holding means for holding the septum within the second chamber 66. This septum holding means is here provided as a generally cylindrically shaped ring-like member 70 and a split septum 72. In a manner presently to be described split septum 72, is penetrable by a cannula, such as a cannula 76 of a connector component 78 (FIG. 5) to gain access to chamber 64. Cannula 76 comprises a plastic cannula having a tapered wall, one end of which interconnects with base 78a and the opposite end of which terminates in a septum penetrating extremity 76a.

As best seen by referring to FIG. 4, ring like member 70 includes an inner wall 71 which defines a septum chamber having an enlarged diameter first sub chamber 70a and a reduced diameter second sub chamber 70b. Sub chambers 70a and 70b are divided by an inwardly extending annular segment 70c, which comprises a part of the septum retaining means of the invention, the character of which will presently be described. The outer surface of ring like member 70 is provided with a circumferentially extending protuberance 80, which comprises a part of the locking means of the invention for locking member 70 in position within lower chamber 66 of body 62. Protuberance 80 is closely received within a groove 82 formed in the interior wall 66a of chamber 66.

Septum 72, which is constructed from a yieldably deformable elastomeric material, includes an enlarged diameter first portion 72a which is closely received within sub chamber 70a of ring like member 70 and a reduced diameter second portion 72b, which is closely received within sub chamber 70b of member 70. As indicated in FIG. 4, septum 72 is provided with a groove 72c, which closely receives annular wall 70c so that the septum is securely retained in position within ring like member 70. For reasons presently to be discussed, ring-like member 70 is preferably constructed from a plastic material that has enough resiliency to allow slight deformation of the skirt portion of the member.

In assembling the sampling site shown in FIG. 4, split septum 72 is mated with ring like member 70 by inserting the elastomeric septum into sub chamber 70a and then pushing it past segment 70c into the secured position shown in the drawings. In this position it is to be noted that clearances provided between the septum and the ring-like member prevent the imposition of either radial or axial forces on the septum by the ring-like member.

After the septum is mated with ring like member 70, the assemblage thus formed is mated with body portion 62 by inserting the assemblage into chamber 66 and exerting a force thereon, which is sufficient to cause protuberance 80 to snap into groove 82. With the closure means thus mated with body portion 62 fluids can be introduced into, or withdrawn from, passageway 44 by means of a cannula, such as cannula 76 (FIG. 5), which is adapted to penetrate septum 72. When penetrated by the cannula, septum 72 functions as a gasket and is self-sealing about the cannula, thereby preventing fluid leakage and air entry to the system while at the same time providing a sterile barrier to atmosphere.

An important feature of the sampling port of the form of the invention shown in FIGS. 1 through 5, is the locking shoulder 86 which functions to lockably engage locking means provided on one or more forms of the connector components of the blood sampling apparatus of the invention the character of which will presently be described. The important blood sampling apparatus of the invention, which includes the blood sampling device as described in the preceding paragraphs, also includes the previously identified first syringe “S” that can be connected to said side port fluid passage of said stopcock assembly as well as a second syringe S-1 that can be connected to the connector components of the apparatus in the manner shown in FIG. 5.

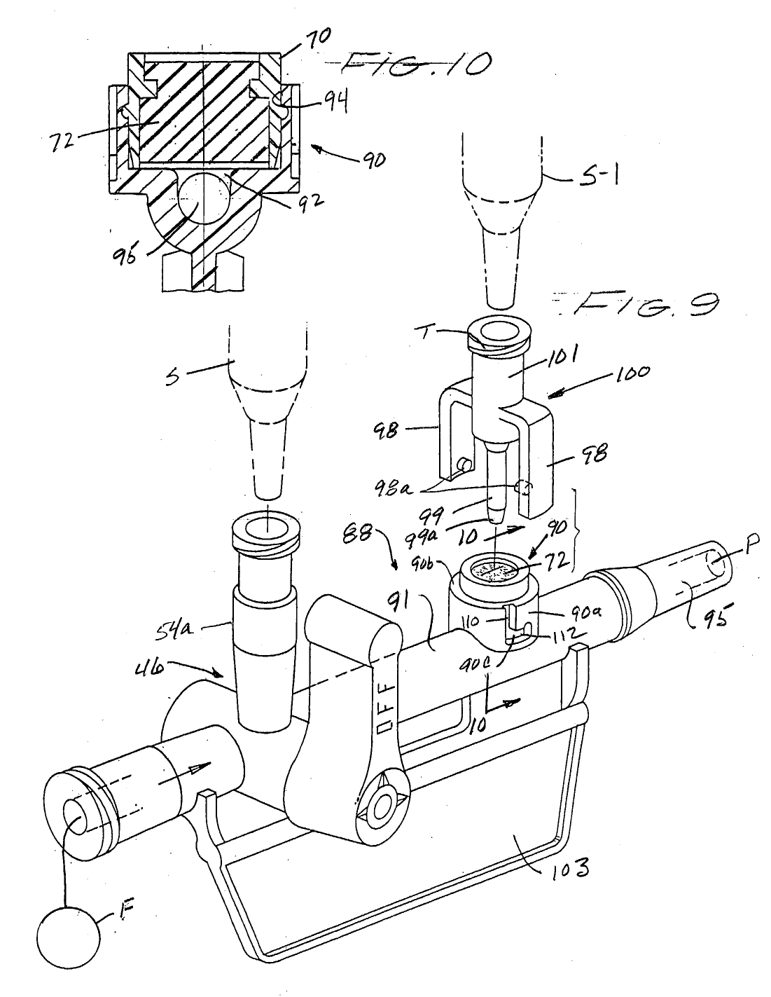

The first syringe “S” forms a part of the first access means of the invention for withdrawing fluid from passageway 44 via the stop cock assembly side port fluid passageway 54a (FIG. 7) in a quantity sufficient to draw blood from the patient past the sampling port assembly 60 so that the first or upper chamber 64 thereof can be accessed to withdraw undiluted blood contained there within using the second syringe S-1. This technique for drawing undiluted blood from the patient will be described in greater detail hereinafter in connection with the description of the method of the invention.

Connector component 78 here comprises a generally cylindrically shaped base portion 78a to which blunt cannula 76 is connected. Integrally formed with base portion 78a are locking means for locking engagement with the sampling port 60. The locking means here comprise a pair of resiliently deformable side members 79, which are interconnected with cylindrical base portion 78a in the manner shown in FIG. 5. Side members 79 extend to either side of cannula 76 and, in their sampling port clamping state, are generally parallel to the longitudinal axis of the cannula. Also forming a part of the connector component are oppositely disposed locking segments 79a which are integrally formed with side members 79 and are adapted to lockably engage shoulder 86 of the sampling port in the manner shown in FIG. 5.

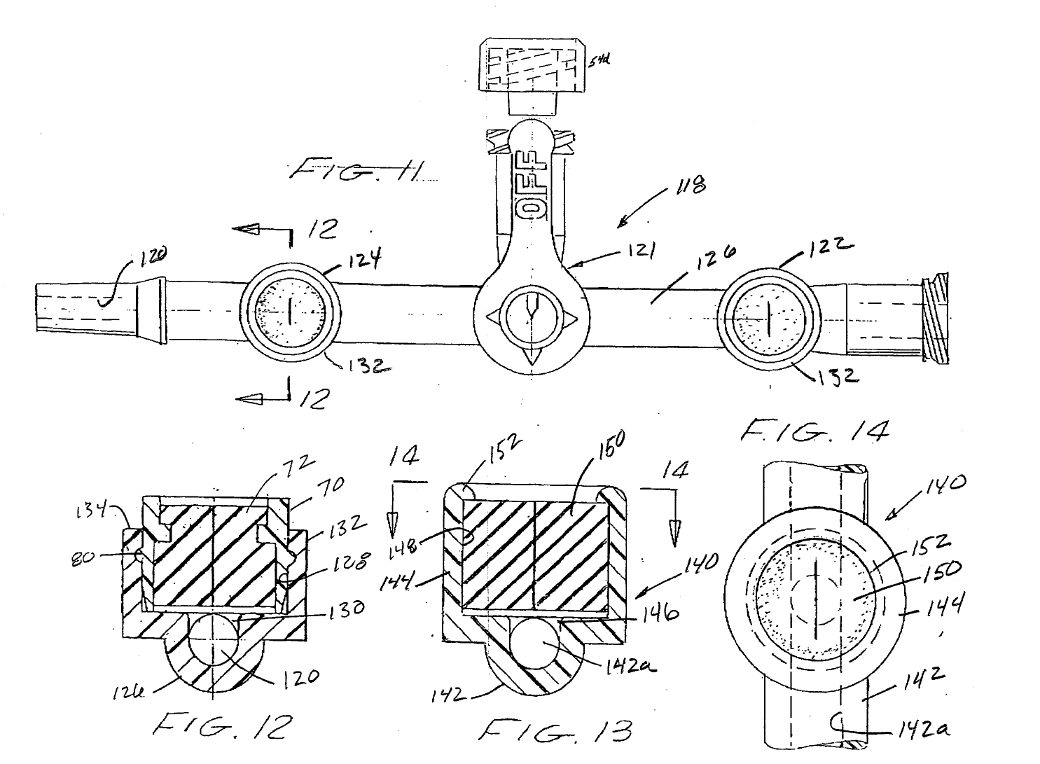

Turning next to FIG. 9, which shows an alternate form of the blood sampling apparatus of the invention, it can be seen that when in use the apparatus is interconnected between a source of fluid “F” such as a heparin solution or the like and the patient “P” from which the blood sample is to be obtained via an intravenous or intraarterial catheter that is invasively connected to the patient (not shown). The apparatus here comprises a unitary blood sampling device 88 that is similar in construction and operation to the unitary blood sampling device shown in FIG. 3 of the drawings and like numerals are used in FIG. 9 to identify like components. Additionally, the blood sampling apparatus comprises first accessing means for accessing the fluid side passage 54a of the stopcock assembly and second accessing means for accessing first chamber 92 of a sampling port assembly 90. The construction and operation of the first and second accessing means of the invention will be described in greater detail hereinafter.

As illustrated in FIGS. 9 and 10, unitary blood sampling device 88 of this latest form of the invention the sampling port assembly 90 is integrally formed with elongated body 91, which body is of the same general construction and as body 42 shown in FIG. 3 of the drawings. As depicted in FIG. 10, the sampling port assembly 90 includes first and second chambers 92 and 94. As in the earlier described blood-sampling device, first chamber 92 is provided with smoothly curved bottom and sidewalls and is in direct communication with fluid passageway 95 of the elongated body 91. The entry and exit paths into chamber 92 of sampling port body 90 smoothly mate tangently to the inside diameter of passageway 95 to provide nonturbulent fluid flow and to significantly minimize dead space volume. As shown in FIG. 9, the outer wall 90a of sampling port body 90b is provided with generally “L” shaped bayonet type grooves 90c which, in a manner presently to be described receive protuberances 98a formed on wings 98 provided on a mating medical connector and 100.

Forming a part of the sampling port assembly of this latest form of the invention is closure means for securely sealing chamber 92 relative to atmosphere. This important closure means is substantially identical in construction and operation to that previously described and comprises a septum holding means provided in the form of a generally cylindrically shaped ring-like member 70 and a split septum 72.

In a manner presently to be described split septum 72, is penetrable by a cannula, such as a cannula 99 of medical connector component 100 to gain access to chamber 92. Cannula 99 comprises a plastic cannula having a tapered wall, one end of which interconnects with body 101 and the opposite end of which terminates in a septum penetrating extremity 99a.

Affixed to elongated, unitary body 92 is outwardly extending gripping means for gripping the blood sampling device using the fingers or for clamping the device to a pole or other structure. This gripping means here comprises an outwardly extending, generally planar member 103 having the configuration best seen in FIG. 9.

The apparatus also includes a conventional syringe S that can be connected to side port fluid passage 54 of a stopcock assembly 46 that connected to body 91 and is identical in construction and operation to that described in connection with FIG. 3 of the drawings. The apparatus further includes the previously mentioned second syringe S-1 that can be connected to the connector component 100 of this latest embodiment of the invention, which component is of a slightly different construction from connector component 78 shown in FIG. 5. More particularly, connector component 100 here comprises a body portion 101, which supports blunt cannula portion 99 that is of sufficient length to extend through split septum 72 and into chamber 92. Body portion 101 is provided with luer type threads “T” proximate its upper end for use in interconnecting the device with an appropriate conduit. Connector component 100 is interconnected with the sampling port by urging cannula 99 into split septum 72 so that protuberances 98a provided on wings 98 of the connector component align with the legs 110 of L shaped groves 90c formed in the exterior wall of sampling body 90. After the cannula portion 98 penetrates septum 72, the connector is rotated in a manner to urge protuberances “P” into portion 112 of the L shaped grooves 90c. Once connected fluids flowing through passageway 95 can be sampled in accordance with a method of the invention and in a manner presently to be described.

As before, passage 95 can be suitably interconnected with a source “S” of liquid such as a parenteral fluid by means of a luer connector or a similar connector known to those skilled in the art. Similarly, passage 95 can be placed in communication with the patient via an intra-arterial/venous line that has been interconnected invasively to the patient.

In accordance with the method of the invention the unitary sampling port shown in FIG. 3, or alternatively the unitary sampling port shown in FIG. 9, is first interposed between the source of fluid “F” and the patient. With the sampling port so positioned, fluid is free to flow from source of fluid “F” through the stopcock assembly 46 in the manner shown in the drawings and onward toward the patient. The method of the invention for obtaining a sample of blood from the patient then comprises the following steps:

1. aseptically wiping surfaces of the side port 54 of the stopcock assembly with a sterile solution such as alcohol or iodine solution;

2. interconnecting syringe S with the side port passageway 54a;

3. rotating the stopcock control handle 65 to the position shown in FIG. 7 where plug 56 blocks the flow of fluid from the source of fluid toward the patient while permitting fluid flow from the patient toward syringe S via passageways 44 and 54a;

4. using syringe S, aspirating all the heparin or flush solution (plus some amount of blood) from the line leading to the patient causing the line, as well as passageways through the body portion to fill completely with the patient's blood,

5. aseptically wiping surfaces of a syringe S-1 and the surfaces of either medical connector 78 or 100 and interconnecting the syringe S-1 with a medical connector in the manner indicated in FIGS. 5 and 9;

6. attaching either connector 78 or connector 100 to one of the sampling port assemblies 60 or 90 so that the cannula pierces the sampling port septum and then using syringe S-1, aspirating the desired volume of blood specimen into the syringe;

7. removing the medical connector from the sampling port assembly;

8. removing the syringe from the medical connector and forwarding the syringe containing the blood sample for laboratory analysis;

9. using the syringe S, reintroducing the fluid contained within the syringe S into passage 44 and into the line leading to the patient;

10. turning the stopcock control handle to the starting position shown in FIGS. 3 and 6 to block fluid flow through passageway 54a and to reopen fluid flow from the source of fluid toward the patient via the passageways through the body portions;

11. removing syringe S from the stopcock assembly; and

12. Reinstituting the flow of fluid toward the patient from the source of fluid to purge the remaining blood in the intra-arterial/venous line back into the patient.

13. rotating the stopcock handle toward the patient thereby opening fluid flow from the source to the side port 54 to permit cleansing flush of the residual blood from the stopcock side port 54.

In accordance with a method of the invention as described in the preceding paragraphs, an uncontaminated and undiluted blood sample can be quickly and easily obtained from the patient without any waste of blood and without opening the system to atmosphere at any time during the sampling process.

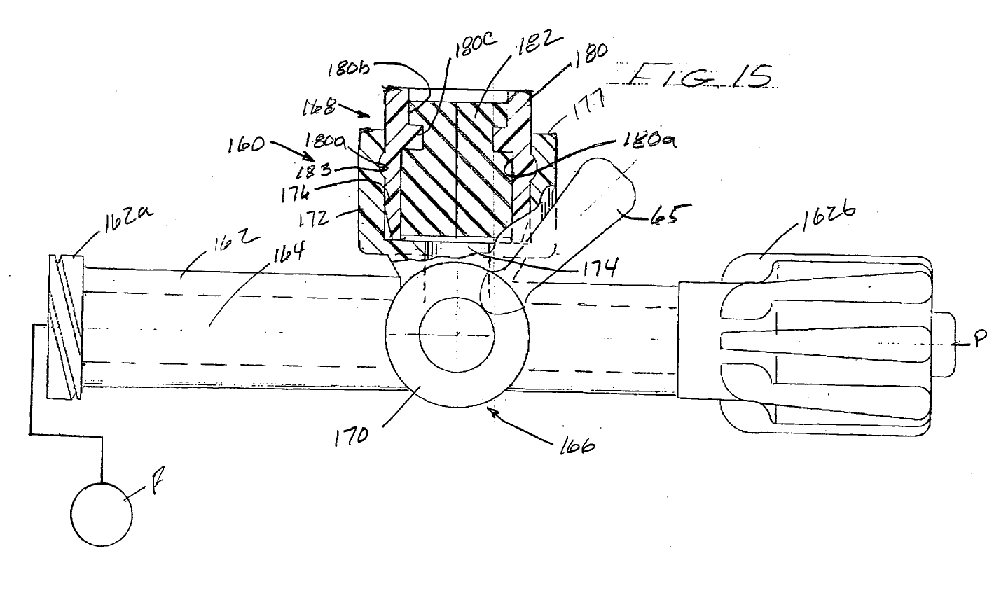

Referring to FIGS. 11 and 12, another form of the blood sampling apparatus of the invention is there shown. This form of the apparatus is similar in some respects to that previously described and like numbers are used in FIGS. 11 and 12 to identify like components. This latest form of the apparatus of the invention comprises a unitary blood sampling device 118 that includes a first and second accessing means for accessing the fluid passage 120 of the apparatus. As shown in FIG. 11, the stopcock assembly may have a deadener port cover 54d on side port 54 as the stopcock will be used to control fluid, but not opened for side port access. Such deadener covers may be used to close the side ports of all of the various stopcock constructions described in the preceding paragraphs when the side ports are not in use. The construction and operation of the first and second accessing means and of the stopcock assembly 121 of this latest form of the invention will be discussed in greater detail hereinafter.

As illustrated in FIGS. 11 and 12 of the drawings, the first and second accessing means are here provided in the form of first and second sampling port assemblies 122 and 124, both of which are integrally formed with an elongated body 126 which body is of similar construction to that shown in FIG. 3 of the drawings. Disposed intermediate these sampling port assemblies is the previously mentioned stopcock assembly 121. As depicted in FIG. 12, each of the identical sampling port assemblies 122 and 124 include upper and lower chambers 128 and 130 respectively. As in earlier described blood sampling devices, lower chamber 130 is provided with smoothly curved sidewalls and is in direct communication with fluid passageway 120 of the elongated body 126. The entry and exit paths into chamber 120 of identical sampling port bodies 132 smoothly mate tangently to the inside diameter of passageway 120 to provide non-turbulent fluid flow and to significantly minimize dead space volume.

Forming a part of each of the identical sampling port assemblies of this latest form of the invention is closure means for securely sealing upper chamber 128 relative to atmosphere. This important closure means is substantially identical in construction and operation to that previously described and comprises a septum holding means provided in the form of a generally cylindrically shaped ring-like member 70 and a split septum 72.

As previously described herein, split septum 72, is penetrable by a cannula, such as a cannula 76 of medical connector component 78 to gain access to chamber 130. As shown in FIG. 5, cannula 76 comprises a plastic cannula having a tapered wall, one end of which interconnects with base portion 78a of the connector component 78 and the opposite end of which terminates in a septum penetrating extremity 76a.

As in the form of the invention shown in FIGS. 1 through 5, each of the sampling port assemblies 122 and 124 has a locking shoulder 134 which functions to lockably engage locking means provided on a connector component such as the component 78 which is shown in FIG. 5.

As earlier described, connector component 78 is provided with side members 79 that extend to either side of cannula 76 and, in their sampling port clamping state, are generally parallel to the longitudinal axis of the cannula. The connector component also includes oppositely disposed locking segments 79a which are integrally formed with side members 79 and are adapted to lockably engage shoulders 134 of the sampling ports 122 and 124.

When the connector component is interconnected with the sampling port assemblies, cannula 76 is urged into piercing engagement with split septum 72 in the manner previously described. After the cannula portion 76 penetrates septum 72, the locking segments 79a of the side members will snap over shoulder 134 of the sampling port assembly thereby into locking the connector component with the sampling port assembly. Once the connector component is so interconnected, fluids flowing through passageway 120 can be sampled in accordance with the latest method of the invention and in a manner next to be described.

As before, passage 120 can be suitably interconnected with a source of liquid “F” such as a parenteral fluid by means of a luer connector or a similar connector known to those skilled in the art. Similarly, passage 120 can be placed in communication with the patient via an intra-arterial/venous line that has been interconnected invasively to the patient.

In accordance with this latest form of the method of the invention, the unitary sampling port 122 is interposed between the source of fluid and the patient in the manner illustrated in FIG. 11. In this configuration, fluid is free to flow from a source of fluid through the stopcock assembly 121, onward toward sampling port 124 and then toward the patient. The method of the invention for obtaining a sample of blood from the patient then comprises the following steps:

1. aseptically wiping surfaces of the side port 54 of the stopcock assembly and the sampling port assembly 122 with a sterile cleansing solution such as alcohol or iodine solution;

2. interconnecting a connector assembly 76 with sampling port assembly 122 so that the cannula pierces the septum 72;

3. rotating the stopcock control handle 12 to the position shown in FIG. 11 where the stopcock blocks the flow of fluid toward the side port permitting fluid flow from the patient toward sampling port assembly 122 via passageway 120;

4. using syringe S, which is interconnected with the connector assembly 76, aspirating all the heparin or flush solution (plus some amount of blood) from the line leading to the patient causing the line, as well as passageway 120, to fill completely with the patient's blood;

5. aseptically wiping surface of sampling port 124 and interconnecting a second medical connector such as a connector 78 with sampling port assembly 124 so that the cannula pierces the septum 72;

6. rotating the stopcock control handle to a closed or midpoint position starting position (see example FIG. 8);

7. using a second syringe interconnecting the syringe with the second connector assembly connected to sampling port assembly 124 and then aspirating a blood specimen into the syringe;

8. removing the medical connector from the sampling port assembly 124;

9. removing the syringe from the medical connector and forwarding the blood sample for laboratory analysis;

10. rotating the stopcock control handle to the open position;

11. using the syringe that is connected to the connector 76 that is affixed to sampling port 122, reintroducing the fluid contained within the syringe S into passage 120 and into the line leading to the patient; and

12. reinstituting the flow of fluid toward the patient from the source of fluid to purge the remaining blood in the intra-arterial/venous line back into the patient.

In accordance with a method of the invention as described in the preceding paragraphs, an uncontaminated and undiluted blood sample can be quickly and easily obtained from the patient without any waste of blood and without opening the system to atmosphere at any time during the sampling process.

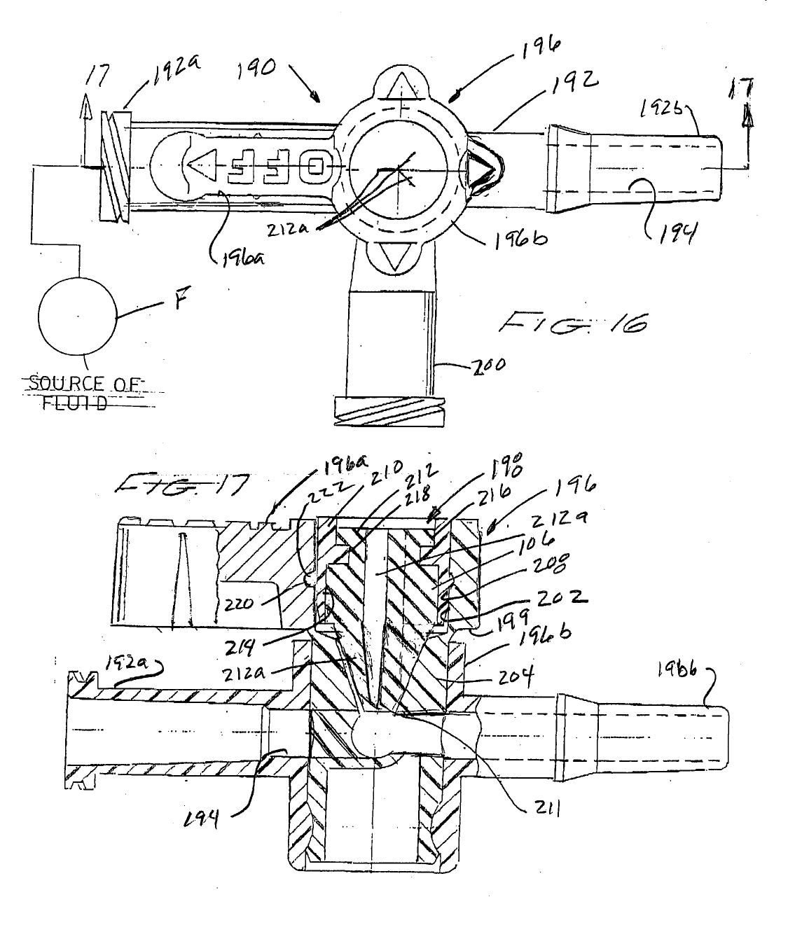

Turning next to FIGS. 13 and 14, an alternate form of the integral blood sampling device of the invention that can be used instead of sampling ports 122 and 124 is there shown. This latest form of sampling port comprises a low dead space sampling port assembly 140 that is integrally formed with an elongated body 142 similar to body 126. As best seen in FIG. 13, this novel sampling port assembly 140 comprises a sampling port body 144 that it is integrally formed with elongated body 142. This latest form of the invention sampling port body 144 includes the first and second chambers 146 and 148 respectively. As illustrated in FIG. 13, the first chamber 146 is provided with smoothly curved bottom and sidewalls and is in direct communication with fluid passageway 142a of elongated body 142. The unitary blood-sampling device of the invention is preferably constructed from a rigid, plastic polycarbonate material, although other plastic materials can be used in its fabrication.

Also forming a part of the sampling port assembly 140 is closure means for securely sealing chamber 146 relative to atmosphere. This important closure means is shown here as a generally cylindrically shaped, split septum 150 that is mounted within chamber 148. Septum holding means for holding the septum in place is provided in the form of a radially inwardly extending lip 152 that is formed on the upper extremity of body 144 by any suitable means such as swaging spin welding, or like deformation. As before split septum 150. In a manner presently to be described split septum 72, is penetrable by a cannula, such as a cannula 76 of a connector component 78 (FIG. 5) to gain access to chamber 146.

Referring next to FIG. 15 of the drawings, still another form of the unitary blood sampling device of the invention is there shown and generally designated by the numeral 160. This alternate form of the device comprises an elongated, unitary plastic body 162 having a fluid passageway 164 there through. Plastic body 162, which is fabricated for use as a single piece, includes, proximate either end, connector portions 162a and 162b. Integrally formed with the elongated body 162 intermediate the first and second ends 162a and 162b is a novel stopcock assembly 166. This unique stopcock assembly is of similar construction and operation to the stopcock shown in FIGS. 3 through 8 save that the syringe accessible side port 54 has been replaced with a sampling port assembly 168 that includes a body portion 170 that has a central chamber that is in communication with fluid passageway 164 of elongated body 162. Body portion 170 of the stopcock assembly further includes an inlet, an outlet and the previously mentioned sampling port assembly 168 each of which is in communication with the central chamber of the stopcock.

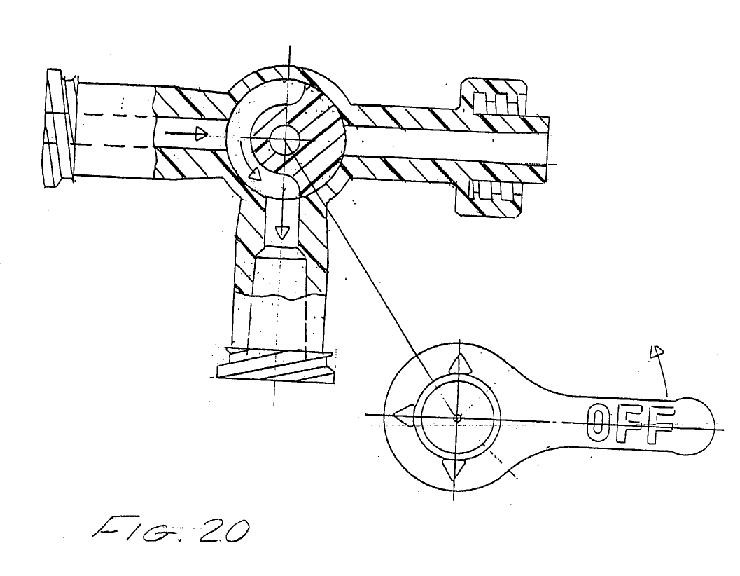

Rotatably mounted within central chamber of the body portion of the stopcock assembly 168 is a flow control member of the character shown in FIGS. 6 through 8 that is provided with a generally “T” shaped fluid passageway for selectively controlling the flow of fluid through the device upon rotation of the flow control member that is mounted within central chamber of the stopcock assembly. As before, rotation of the flow control member is accomplished by rotating the stopcock handle 65 in the manner illustrated in FIGS. 6 through 8. Alternatively, a semicircular groove of the character shown in FIG. 18 can be used to control fluid flow through the device.

The novel sampling port assembly 168 that takes the place of the side port 54 of the conventional stopcock is integrally formed with elongated body 162 and comprises a sampling port body 172 that is integrally formed with stopcock body portion 170. Sampling port body 172 includes the first and second chambers 174 and 176 respectively.

Also forming a part of the sampling port assembly is closure means for securely sealing chamber 174 relative to atmosphere. This closure means is generally similar to that previously described herein in connection with the embodiments of the invention shown in FIGS. 3 through 12. As shown in FIG. 12, the closure assembly here comprises a septum holding means for holding the septum within the second chamber 176. This septum holding means is here provided as a generally cylindrically shaped, ring-like member 180 and a split septum 182. As before, septum 182 is penetrable by a cannula, such as a cannula 76 of a connector component 78 (FIG. 5) to gain access to chamber 174. Ring like member 180 includes an inner wall 180a which defines a septum chamber having an enlarged diameter chamber 180a and a reduced diameter second subchamber 180b. Subchambers 180a and 180b are divided by an inwardly extending annular segment 180c, which comprises a part of the septum retaining means of the invention. The outer surface of ring-like member 180 is provided with a circumferentially extending protuberance 180a, which comprises a part of the locking means of the invention for locking member 180 in position within body 172. Protuberance 180a is closely received within a groove 183 formed in the interior wall of body 172.

Sampling port 168 of this latest form of the invention also includes a locking shoulder 177 which functions to lockably engage locking means provided on a connector component such as that shown in FIG. 5. In this latest embodiment, the connector component takes the place of the syringes “S” to enable the withdrawal of fluid from passageway 164 in a quantity sufficient to draw blood from the patient for sampling purposes in the manner previously described.

In accordance with an alternate method of the invention using the apparatus shown in FIG. 15, the apparatus is first interposed between the source of fluid “F” and the patient “P”. With the sampling port so positioned and with the stopcock in an open position, fluid is free to flow from the source of fluid “F” through the stopcock assembly and onward toward the patient. The method of the invention for obtaining a sample of blood from the patient then comprises the following steps:

1. aseptically wiping surfaces of the sampling port 168 of the stopcock assembly with a sterile solution such as alcohol or iodine solution;

2. interconnecting a connector component such as component 78 with the sampling port;

3. rotating the stopcock control handle 65 to a position where the stopcock plug blocks the flow of fluid from the source of fluid toward the patient while permitting fluid flow from the patient toward the sampling port;

4. using syringe S-1, aspirating all the heparin or flush solution (plus some amount of blood) from the line leading to the patient causing the line, as well as passageways through the body portion to fill completely with the patient's blood;

5. using a second syringe interconnecting the syringe with connector assembly 78 that is connected to sampling port assembly 168 and then aspirating a blood specimen into the syringe;

6. removing the medical connector from the sampling port assembly 168;

7. removing the second syringe from the medical connector and forwarding the blood sample for laboratory analysis;

8. rotating the stopcock control handle to the open position;

9. using syringe S-1, reintroducing the fluid contained within the syringe S into the line leading to the patient; and

10. reinstituting the flow of fluid toward the patient from the source of fluid to purge the remaining blood in the intra-arterial/venous line back into the patient.

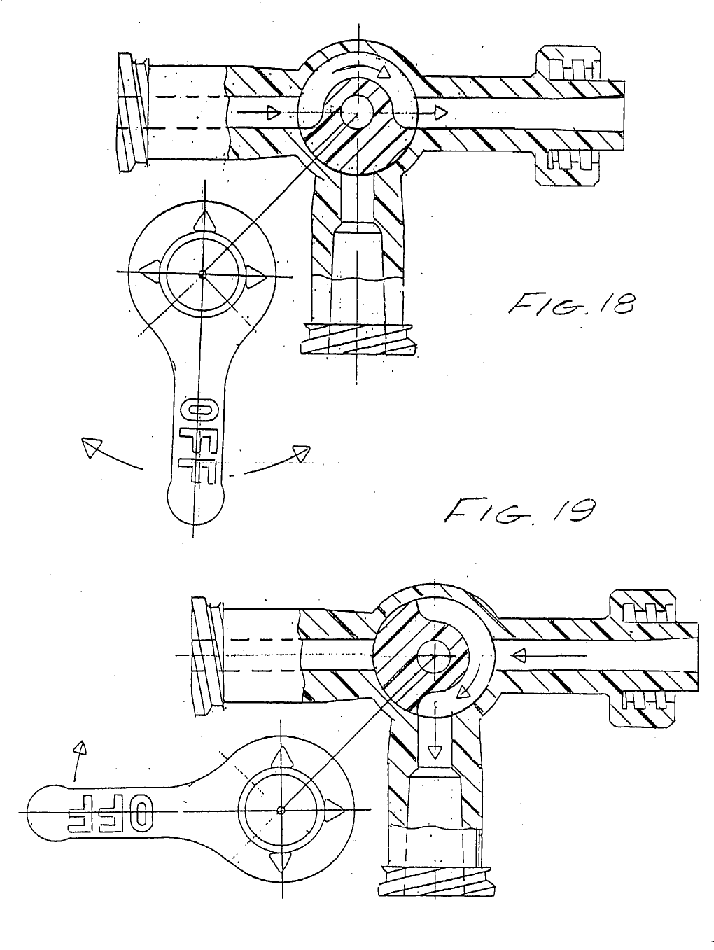

Turning to FIGS. 16 and 17 of the drawings, yet another form of the unitary blood sampling device of the invention is there shown and generally designated by the numeral 190. This alternate form of the device comprises an elongated, unitary plastic body 192 having a fluid passageway 194 there through. Plastic body 192, which is fabricated for use as a single piece, includes, proximate either end, connector portions 192a and 192b. Integrally formed with the elongated body 162 intermediate the first and second ends 162a and 162b is a novel stopcock assembly 196. Once again, this unique stopcock assembly is somewhat of similar construction and operation to the stopcock shown in FIGS. 3 through 8 save that the novel sampling port assembly 198 is mounted in the handle portion 196a of the stopcock assembly 196. As before, a syringe accessible side port 200 having a syringe receiving tapered bore is connected to the body portion 196b of the stopcock assembly. Sampling port assembly 198 has a central chamber 202 that is in communication with fluid passageway 194 of elongated body 192. Body portion 196b of the stopcock assembly further includes an inlet and an outlet each of which is in communication with the central chamber of the stopcock.

Rotatably mounted within central chamber of the body portion of the stopcock assembly 198 is a flow control member 204 of the character shown in FIGS. 6 through 8 that is provided with a generally “T” shaped fluid passageway for selectively controlling the flow of fluid through the device upon rotation of the flow control member. As before, rotation of the flow control member is accomplished by rotating the stopcock handle 196a in the manner illustrated in FIGS. 6 through 8.

The novel sampling port assembly 198 that is uniquely mounted with the stopcock handle portion includes the first and second chambers 208 and 211 respectively.

Also forming a part of the sampling port assembly is closure means for securely sealing chamber 208 relative to atmosphere. This closure means is generally similar to that previously described herein in connection with the embodiments of the invention shown in FIGS. 3 through 12. As shown in FIG. 17, the closure assembly here comprises a septum holding means for holding the septum within the second chamber 208. This septum holding means is here provided as a generally cylindrically shaped, ring-like member 210 and a split septum 212 having a plurality of slits 212a (FIG. 17). As before, septum 212 is penetrable by a cannula, such as a cannula 76 of a connector component 78 (FIG. 5) to gain access to chamber 211. However, in this latest embodiment of the invention, the septum 212 includes a tapered lower portion 212a that extends into the flow control member in the manner shown in FIG. 17. Ring like member 210 includes an inner wall 210a which defines a septum chamber having an enlarged diameter chamber 214 and a reduced diameter second subchamber 216. Subchambers 214 and 216 are divided by an inwardly extending annular segment 218 which comprises a part of the septum retaining means of the invention. The outer surface of ring-like member 210 is provided with a circumferentially extending protuberance 220, which comprises a part of the locking means of the invention for locking member 210 in position within the handle portion. Protuberance 220 is closely received within a groove 222 formed in the interior wall of handle member 196a.

Sampling port 198 of this latest form of the invention also includes a locking shoulder 199 which functions to lockably engage locking means provided on a connector component such as that shown in FIG. 5 to enable the withdrawal of fluid from passageway 194.

In accordance with an alternate method of the invention using the apparatus shown in FIGS. 16 and 17, the apparatus is first interposed between the source of fluid “F” and the patient “P”. With the sampling port so positioned and with the stopcock in an open position, fluid is free to flow from the source of fluid “F” through the stopcock assembly and onward toward the patient. The method of the invention for obtaining a sample of blood from the patient then comprises the following steps:

1. aseptically wiping surfaces of the sampling port 198 of the stopcock assembly with a sterile solution such as alcohol or iodine solution;

2. interconnecting a connector component such as component 78 with the sampling port;

3. rotating the stopcock control handle 196a to the position shown in FIG. 17 where the stopcock plug blocks the flow of fluid from the source of fluid toward the patient while permitting fluid flow from the patient toward the sampling port;

4. using syringe S-1 that is connected to side port 200, aspirating all the heparin or flush solution (plus some amount of blood) from the line leading to the patient causing the line, as well as passageways through the body portion to fill completely with the patient's blood;

5. using a second syringe interconnecting the syringe with the second connector assembly that is connected to stopcock assembly 196 and then aspirating a blood specimen into the syringe;

6. removing the medical connector from the stopcock assembly 196;

7. removing the second syringe from the medical connector and forwarding the blood sample for laboratory analysis;

8. rotating the stopcock control handle to the open position;

9. using syringe that is connected to port 200, reintroducing the fluid contained within the syringe S into the line leading to the patient; and

10. reinstituting the flow of fluid toward the patient from the source of fluid to purge the remaining blood in the intra-arterial/venous line back into the patient.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following documents.

I claim:

1. A unitary sampling device for interconnection invasively with a patient for obtaining a blood sample from the patient, said unitary sampling device being closed to atmosphere and comprising:
(a) an elongated body having a fluid passageway there through, said fluid passageway having first and second ends;
(b) a stopcock assembly connected to said elongated body intermediate said first and second ends, said stopcock assembly comprising:
(i) a stopcock body integrally formed with said elongated body and having bottom and sidewalls defining a central chamber in communication with said fluid passageway of said elongated body, said stopcock body further including inlet, outlet and side port fluid passages in communication with said central chamber; and
(ii) flow control member having a “T” shaped fluid passageway for selectively controlling the flow of fluid through said inlet, outlet and side port fluid passageways upon rotation of said flow control member within said central chamber;
(c) a sampling port assembly connected to said elongated body intermediate said stopcock assembly and said second end of said elongated body, said sampling port assembly comprising:
(i) a sampling port body integrally formed with said elongated body and having first and second in chambers, said first chamber being in communication with said fluid passageway of said elongated body; and
(ii) closure means disposed within said second chamber for sealing said first chamber relative to atmosphere.

2. The sampling device as defined in claim 1 in which said bottom and sidewalls of said central chamber of said stopcock assembly smoothly mate with said fluid passageway of said elongated body to provide nonturbulent laminar fluid flow and to substantially eliminate dead space volume.

3. The sampling device as defined in claim 1 in which said closure means of said sampling port assembly comprises:
(a) a septum pierceable by a cannula to gain access to said first chamber of said sampling port; and
(b) septum holding means for holding said septum within said second chamber, said septum holding means including a generally ring shaped member telescopically received within said second chamber, said member having an inner wall defining a septum chamber and including septum retaining means for retaining said septum within said septum chamber, said retaining means comprising a segment extending inwardly from said inner wall into said septum chamber.

4. The sampling device as defined in claim 3 in which said septum comprises a split septum.

5. The sampling device as defined in claim 3, further including locking means for locking said closure means within said second chamber.

6. A unitary sampling device for interconnection invasively with a patient for obtaining a blood sample from the patient, said unitary sampling device being closed to atmosphere and comprising:
(a) an elongated body having a fluid passageway there through, said fluid passageway having first and second ends;
(b) a stopcock assembly connected to said elongated body intermediate said first and second ends, said stopcock assembly comprising:
(i) a stopcock body integrally formed with said elongated body and having bottom and sidewalls defining a central chamber in communication with said fluid passageway of said elongated body, said stopcock body further including inlet, outlet and side port fluid passages in communication with said central chamber; and
(ii) flow control member having a “T” shaped fluid passageway for selectively controlling the flow of fluid through said inlet, outlet and side port fluid passageways upon rotation of said flow control member within said central chamber;
(c) a sampling port assembly connected to said elongated body intermediate said stopcock assembly and said second end of said elongated body, said a sampling port comprising:
(i) a sampling port body integrally formed with said elongated body and having first and second in chambers, said first chamber being in communication with said fluid passageway of said elongated body; and
(ii) closure means disposed within said second chamber for sealing said first chamber relative to atmosphere, said closure means comprising:
a. a septum pierceable by a cannula to gain access to said first chamber of said sampling port; and
b. septum holding means for holding said septum within said second chamber, said septum holding means including a generally ring shaped member telescopically received within said second chamber.

7. The sampling device as defined in claim 6, in which said generally ring shaped member has an inner wall defining a septum chamber and further includes septum retaining means for retaining said septum within said septum.

8. The sampling device as defined in claim 7 in which said septum retaining means comprises a segment extending inwardly from said inner wall of said generally ring shaped member into said septum chamber.

9. A blood sampling apparatus for interconnection invasively with a patient for obtaining a blood sample from the patient, said blood sampling apparatus comprising:
(a) a unitary sampling device comprising:
(i) an elongated body having a fluid passageway there through, said fluid passageway having first and second ends;
(ii) a stopcock assembly connected to said elongated body intermediate said first and second ends, said stopcock assembly comprising:
a. a stopcock body integrally formed with said elongated body and having bottom and sidewalls defining a central chamber in communication with said fluid passageway of said elongated body, said stopcock body further including inlet, outlet and side port fluid passages in communication with said central chamber; and
b. a flow control member having a fluid passageway for selectively controlling the flow of fluid through said inlet, outlet and side port fluid passageways upon rotation of said flow control member within said central chamber;
(iii) a sampling port assembly connected to said elongated body intermediate said stopcock assembly and said second end of said elongated body, said a sampling port assembly comprising:
a. a sampling port body integrally formed with said elongated body and having first and second chambers, said first chamber being in communication with said fluid passageway of said elongated body; and
b. closure means disposed within said second chamber for sealing said first chamber relative to atmosphere;
(iv) first accessing means for accessing said fluid passageway of said elongated body via said stopcock assembly; and
(v) second accessing means for accessing said fluid passageway of said elongated body via said sampling port assembly.

10. The apparatus as defined in claim 9 in which unitary sampling device further includes gripping means connected to said elongated body for gripping said unitary sampling device.

11. The apparatus as defined in claim 9 in which said closure means of said sampling port comprises:
(a) a septum pierceable by a cannula to gain access to said first chamber of said sampling port; and
(b) septum holding means for holding said septum within said second chamber.

12. The apparatus as defined in claim 11 in which said septum holding means comprises a generally ring shaped member telescopically received within said second chamber, said member having an inner wall defining a septum chamber and including septum retaining means for retaining said septum within said septum chamber, said retaining means comprising a segment extending inwardly from said inner wall into said septum chamber.

13. The apparatus as defined in claim 11 in which said septum holding means comprises a radially inwardly extending lip formed on said sampling port assembly.

14. The apparatus as defined in claim 11 in which said first accessing means comprises a conventional syringe.

15. The apparatus as defined in claim 11 in which said second accessing means comprises a medical connector for releasable interconnection with said sampling port assembly.

16. The apparatus as defined in claim 15 in which said medical connector comprises:
(a) a base;
(b) a cannula connected to said base and extending there from; and
(c) locking means connected to said base and extending there from for locking engagement with said sampling port.

17. The apparatus as defined in claim 16 in which said sampling port body of said sampling port assembly is provided with generally “L” shaped bayonet type grooves and in which said locking means comprises a pair of side members connected to said base, each of said side members having protuberances receivable within said generally “L” shaped bayonet type grooves.

18. The apparatus as defined in claim 16 in which said locking means comprises a pair of resiliently deformable side members.

19. The apparatus as defined in claim 18 further including locking segments that are integrally formed with resiliently deformable side members.

20. A unitary sampling device for interconnection invasively with a patient for obtaining a blood sample from the patient, said unitary sampling device being closed to atmosphere and comprising:
(a) an elongated body having a fluid passageway there through, said fluid passageway having first and second ends; and
(b) a stopcock assembly connected to said elongated body intermediate said first and second ends, said stopcock assembly comprising:
(i) a stopcock body integrally formed with said elongated body and having bottom and sidewalls defining a central chamber in communication with said fluid passageway of said elongated body, said stopcock body further including inlet, outlet and side port fluid passages in communication with said central chamber;
(ii) flow control member having a fluid passageway for selectively controlling the flow of fluid through said inlet, outlet and side port fluid passageways upon rotation of said flow control member within said central chamber;
(iii) a sampling port assembly connected to said stopcock body proximate said side port fluid passage, said sampling port assembly comprising:
a. a sampling port body having first and second in chambers, said first chamber being in communication with said side port fluid passageway of said stopcock body; and
b. closure means disposed within said second chamber for sealing said first chamber relative to atmosphere.

21. The sampling device as defined in claim 20 in which said closure means of said sampling port assembly comprises:
(a) a septum pierceable by a cannula to gain access to said first chamber of said sampling port; and
(b) septum holding means for holding said septum within said second chamber, said septum holding means including a generally ring shaped member telescopically received within said second chamber, said member having an inner wall defining a septum chamber and including septum retaining means for retaining said septum within said septum chamber, said retaining means comprising a segment extending inwardly from said inner wall into said septum chamber.

22. The sampling device as defined in claim 21, further including locking means for locking said closure means within said second chamber.

23. The sampling device as defined in claim 21 in which said fluid passageway of said flow control member is generally “T” shaped.

24. The sampling device as defined in claim 21 in which said fluid passageway of said flow control member is curved.

25. A unitary sampling device for interconnection invasively with a patient for obtaining a blood sample from the patient, said unitary sampling device being closed to atmosphere and comprising:
(a) an elongated body having a fluid passageway there through, said fluid passageway having first and second ends;
(b) a stopcock assembly connected to said elongated body intermediate said first and second ends, said stopcock assembly comprising:
(i) a stopcock body integrally formed with said elongated body and having bottom and sidewalls defining a central chamber in communication with said fluid passageway of said elongated body, said stopcock body further including inlet, outlet and side port fluid passages in communication with said central chamber; and
(ii) flow control member including a handle portion and a body portion having a “T” shaped fluid passageway for selectively controlling the flow of fluid through said inlet, outlet and side port fluid passageways upon rotation of said flow control member within said central chamber, said handle portion having a chamber;
(iii) closure means disposed within said chamber of said handle for sealing said chamber of said handle relative to atmosphere, said closure means comprising:
a. a septum pierceable by a cannula to place said cannula in communication with said inlet, outlet and side port fluid passageway of said body portion of said flow control member; and
b. septum holding means for holding said septum within said second chamber, said septum holding means including a generally ring shaped member telescopically received within said chamber of said handle portion.

26. The sampling device as defined in claim 25, in which said generally ring shaped member has an inner wall defining a septum chamber and further includes septum retaining means for retaining said septum within said septum chamber.

27. A method of obtaining a blood sample from a patient in conjunction with a unitary sampling device which is interconnected invasively with the patient by a catheter inserted into either a vein or artery of the patient, the unitary sampling device comprising an elongated body having a fluid passageway there through, the fluid passageway having a first end of connected to a source of fluid and a second end connected to the catheter, a stopcock assembly connected to the elongated body intermediate the first and second ends, said stopcock assembly having a stopcock body integrally formed with the elongated body of the unitary sampling device and a sampling port assembly connected to the elongated body intermediate the stopcock assembly and the second end of the elongated body, the sampling port assembly having a sampling port body integrally formed with the elongated body of the unitary sampling device, said method comprising the steps of:
(a) accessing the stopcock assembly;
(b) removing the fluid contained within the catheter and within the portion of the fluid passageway disposed between said stopcock assembly and the catheter;
(c) drawing undiluted blood from the patient to fill the system between the catheter and the stopcock assembly;
(d) accessing the sampling port assembly; and
(e) drawing a predetermined volume of undiluted blood.

28. A method of obtaining a blood sample from a patient in conjunction with a unitary sampling device which is interconnected invasively with the patient by a catheter inserted into either a vein or artery of the patient, the unitary sampling device comprising an elongated body having a fluid passageway there through, the fluid passageway having a first end of connected to a source of fluid for providing fluid flow through the fluid passageway and a second end connected to the catheter, a stopcock assembly connected to the elongated body intermediate the first and second ends, said stopcock assembly having a stopcock body integrally formed with the elongated body of the unitary sampling device, a syringe mateable with the stopcock assembly to access the fluid passageway and a sampling port assembly connected to the elongated body intermediate the stopcock assembly and the second end of the elongated body, the sampling port assembly having a sampling port body integrally formed with the elongated body of the unitary sampling device, said method comprising the steps of:
(a) using the stopcock assembly, restricting fluid flow from the source of fluid toward the catheter;
(b) mating the syringe with the stopcock assembly and using the stopcock assembly and the syringe, accessing the fluid passageway to draw into the syringe a volume of fluid comprising the fluid contained within the fluid passageway between the catheter and the stopcock assembly and to draw blood from the patient to fill the fluid passageway between the stopcock assembly and the catheter;
(c) using the sampling port assembly, accessing the fluid passageway to draw from the patient a predetermined volume of undiluted blood; and
(d) using the stopcock assembly and the syringe, introducing into the fluid passageway the volume of fluid drawn into the syringe.

29. The method as defined in claim 28 including a further step of using the stopcock assembly, re-establishing fluid flow from the source of fluid toward the catheter.

30. The method as defined in claim 29 including a further step of removing the syringe from the stopcock assembly.
